(19) United States
(12) Patent Application Publication
(10) Pub. No.: US 2025/0302727 A1
(43) Pub. Date: Oct. 2, 2025

(54) COSMETIC USE OF A COMBINATION OF IMMORTELLE EXTRACTS TO REDUCE THE VISIBILITY OF SKIN PORES

(71) Applicant: LABORATOIRES M&L, MANOSQUE (FR)

(72) Inventors: PIERRE-NICOLAS URBAIN, VALENSOLE (FR); MARION HARO, ORAISON (FR); VIRGINIE ROUQUET, MANOSQUE (FR); CHARLINE COMBES, MANOSQUE (FR)

(21) Appl. No.: 18/849,670

(22) PCT Filed: Mar. 14, 2023
(86) PCT No.: PCT/EP2023/056476

§ 371 Date: Sep. 23, 2024

(30) Foreign Application Priority Data
Mar. 24, 2022 (FR) FR2202610

(51) Int. Cl. A61K 8/92; A61K 8/37; A61K 8/60; A61K 8/9789; A61Q 19/00; A61Q 19/08

(52) U.S. Cl. CPC A61K 8/922; A61K 8/375; A61K 8/602; A61K 8/9789; A61Q 19/00; A61Q 19/08

(57) ABSTRACT

The present invention relates to the cosmetic use of a combination of extracts of immortelle, namely an essential oil of immortelle of the species Helichrysum italicum and a subcritical water extract of distillation residue of immortelle of the species Helichrysum italicum, in order to reduce the visibility of skin pores and/or tighten skin pores.

### FIELD OF THE INVENTION

The present invention relates to the cosmetic use of a combination of extracts of immortelle, namely an essential oil of immortelle and a subcritical water extract of distillation residue of immortelle, in order to reduce the visibility of skin pores and/or tighten skin pores.

### BACKGROUND OF THE INVENTION

Pores are small openings at the surface of the skin, the physiological role of which is to make possible the excretion of sweat and sebum.

They are in communication with the sebaceous glands, often via a hair follicle ending in a single and very short excretory duct. The sebaceous glands are located in the dermis, which is a connective tissue composed of an extracellular matrix (ECM) including fibrous proteins (collagen, elastin), glycosaminoglycans and proteoglycans and cells such as fibroblasts. The excretory duct of the sebaceous glands reaches the surface of the epidermis. The dermis and the epidermis are separated from each other by a basement membrane which constitutes the dermo-epidermal junction. The latter is a complex structure of which type IV collagen is a predominant constituent. With age, the degradation of type I collagen, which is the predominant constituent of the ECM, brings about a degradation of the latter which plays its supporting role less well, which results in a deformation of the excretory ducts and thus in a dilation of the pores. In addition, the decrease with age in the content of type IV collagen brings about a detrimental change in the basement membrane, which tends to flatten, and induces a widening of the pores, which become more visible.

The dilation of the pores can also have other causes than skin aging and in particular can originate from inadequate hygiene, climatic or ethnic factors, or an excess of sebum.

In the latter case, the pores are more dilated on the face and in particular on the T zone (forehead, nose, chin), in particular on the area of the nose, and can be accentuated by an increase in the width of the conical portion around the pore and/or a parakeratosis state of the stratum corneum at this conical portion, itself sometimes linked to an excessive secretion of sebum and of unsaturated fatty acids.

This imperfection is often perceived negatively, insofar as it does not convey an image of healthy skin.

It will therefore be understood that it would be useful to have available a means making it possible to thus reduce the unaesthetic dilation of the skin pores.

To this end, camouflage techniques have already been proposed, such as, for example, the use of make-up compositions which can contain polymers or inorganic powders. However, these compositions tend to block the pores, thus generating other imperfections, such as blackheads.

Another solution consists in applying, to the skin, astringent lotions which can contain aluminum salts, aqueous/alcoholic solutions, combinations of retinoids and of hydroxycarboxylic acids, or also inhibitors of parakeratosis, such as glycine derivatives. However, these substances are known to be irritating to the skin.

To reduce the visibility of the pores, several medical techniques have furthermore been proposed, including the use of pulsed light, medical light-emitting diodes (LEDs), lasers and chemical peels.

The need thus remains to have available a means for visibly reducing the pores, which is as effective as those known but which does not exhibit their disadvantages, that is to say in particular which does not bring about skin irritation.

The Applicant Company has already proposed compositions based on ingredients of natural origin which are effective for this purpose.

Thus, the document FR 3 003 167 (M&L) describes a cosmetic composition including three different extracts of immortelle, including an essential oil and an aqueous extract, and also an essential oil of myrtle. This composition is in particular judged by a panel of users to be effective in reducing the appearance of pores.

The document US2011/159125 (Avon) for its part describes a CGRP stimulator, which can be chosen from numerous plant extracts, including essential oils, in particular an extract of Helichrysum gymnocephalum. These extracts are presented as being useful as anti-aging agents, in particular for reducing the size of pores. However, it is not demonstrated that an essential oil of Helichrysum gymnocephalum actually makes it possible to reduce the size of pores. In addition, such an essential oil differs from that employed in the present invention by the absence of neryl acetate (S. Afoulous et al., Molecules, 2011, 16, 8273-8291), which is, on the contrary, present in an amount of at least 30% in the essential oil of Helichrysum italicum, in particular of Corsican origin (A. Bianchini et al., Flavour and Fragrance Journal, 2001, 16, 30-34). Similarly, the document CN109010520 discloses an antiacne composition exhibiting an astringent effect and containing many ingredients, including an essential oil of immortelle which is used to reduce scars because of its ability to regenerate cells.

Nevertheless, the need remains to have available active ingredients of natural origin which are effective in visibly reducing pores.

The Applicant Company has now demonstrated entirely surprisingly that a particular combination of extracts of immortelle makes it possible to achieve this objective, even at low concentrations, while being well tolerated.

### SUMMARY OF THE INVENTION

A subject matter of the invention is specifically the cosmetic use of a combination of an essential oil of immortelle of the species Helichrysum italicum and of an extract of distillation residue of immortelle of the species Helichrysum italicum obtained by extraction of the distillation residue with subcritical water, in order to reduce the visibility of skin pores and/or to tighten skin pores.

Another subject matter of the invention is a cosmetic method for reducing the visibility of skin pores and/or tightening skin pores, comprising the topical application to the skin of a cosmetic composition including the combination of an essential oil of immortelle of the species Helichrysum italicum and of an extract of distillation residue of immortelle of the species Helichrysum italicum obtained by extraction of the distillation residue with subcritical water.

### DETAILED DESCRIPTION

The present invention employs an essential oil of immortelle and an extract of distillation residue of immortelle, both resulting from the species Helichrysum italicum.

An essential oil of Helichrysum italicum of the subspecies italicum or microphyllum and/or of Corsican origin is preferentially used.

The term “essential oil” is understood to mean, in this description, the product of hydrodistillation or of steam distillation of the volatile organic compounds present in any part of the immortelle or of the whole plant, and more particularly in its aerial parts, such as, for example, its flowers or flowering heads (that is to say, flowers and a few centimeters of stems). In these processes, the odorous molecules contained in the plant are released and mechanically entrained with steam, which is either formed by boiling of the water to which the plant is added (conventional hydrodistillation), or formed by boiling of the water contained in the plant (microwave hydrodistillation, optionally under vacuum), or also supplied by a boiler and circulated through the plant (steam distillation). The distillate is subsequently decanted to separate the essential oil used according to the invention from the hydrosol (or floral water). The solid residue which constitutes the distillation residue can be recovered and treated to prepare the extract of distillation residue of immortelle, which is also used in the present invention.

The essential oil of Helichrysum italicum of Corsican origin preferentially used according to the invention contains, as main constituent, neryl acetate, which generally represents at least 30% by weight of the total constituents of the essential oil, as measured by gas chromatography coupled with an analysis by mass spectrometry (GC-MS).

The essential oil of immortelle is combined, in this composition, with an extract of distillation residue of immortelle. The term “distillation residue” is understood to mean the solid residue remaining after hydrodistillation or steam distillation of any part of the immortelle, preferentially of its flowering heads, and separation of the essential oil produced and of the hydrosol produced.

This extract of distillation residue of immortelle is characterized in that it includes flavonol heterosides, the B ring of which is dihydroxylated.

Flavonoids constitute a class of polyphenols including the following backbone:

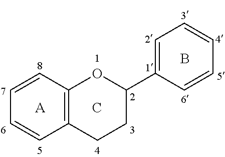

They can themselves be subdivided into different categories according to the carbon of the C ring to which the B ring is attached, the presence or absence of one or more unsaturations in the C ring and/or of an oxo group in the 4 position and/or of a hydroxyl group in the 3 position of the C ring.

Flavonols constitute a category of flavonoids which corresponds specifically to the following structure:

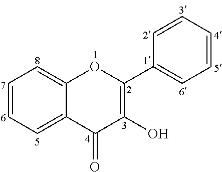 [SMILES: O=c1c(O)c(-c2ccccc2)oc2ccccc12]

These include in particular flavonols which are unsubstituted on the B ring, such as baicalein, flavonols which are monohydroxylated on the B ring, such as kaempferol, and flavonols which are dihydroxylated on the B ring, such as quercetin and quercetagetin.

The Applicant Company has demonstrated, by gas chromatography-mass spectrometry, that the extract of distillation residue of immortelle according to the invention is characterized by the presence of flavonol heterosides, the B ring of which is dihydroxylated, which do not appear in other aqueous extracts, obtained in particular by using water at ambient temperature and pressure as sole extraction solvent. These flavonol heterosides generally comprise at least one quercetagenin heteroside, in particular an O-hexoside, at least one quercetin heteroside, in particular a quercetin O-hexoside-O-rhamnoside, or their mixtures. Typically, the extract of distillation residue of immortelle used according to the invention includes at least 30 μg/ml of flavonol heterosides, the B ring of which is dihydroxylated.

The extract of distillation residue of immortelle used according to the invention additionally includes chlorogenic acid and leontopodic acid B, in a ratio by weight of flavonol heterosides, the B ring of which is dihydroxylated, to chlorogenic acid of between 0.05:1 and 1:1, preferably from 0.1:1 to 0.5:1, and in a ratio by weight of flavonol heterosides, the B ring of which is dihydroxylated, to leontopodic acid B of between 0.05:1 and 1:1, preferably between 0.1:1 and 0.5:1.

In addition, the extract of distillation residue of immortelle used according to the invention generally includes less than 100 ppm of volatile compounds capable of being detected by gas chromatography-mass spectrometry. These volatile compounds include more than 30% of ketones, generally more than 35% of ketones, in particular more than 25% of diketones, generally more than 30% of diketones. The diketones comprise in particular italidiones, 4-methyl-3,5-decanedione, 2,4-dimethylheptane-3,5-dione, 3,5-dimethyloctan-4,6-dione, 7-ethyl-4,6-undecanedione and ethyldodecanedione. The volatile compounds of the extract of distillation residue according to the invention also include less than 2%, generally less than 1%, of nerol. The extract of distillation residue of immortelle can be obtained by a process comprising the extraction with subcritical water of distillation residue of immortelle. This process is advantageously carried out in dynamic mode, by percolation of the water in the subcritical state through the distillation residue arranged in an autoclave, generally of tubular type. The distillation residue can be in dry or fresh form and may or may not be ground. Dried and ground distillation residue is advantageously used. The passage time τ of the subcritical water is preferably between 10 and 20 minutes, preferentially between 11 and 17 minutes, for example approximately 15 minutes, as measured according to the following formula: τ=V/Q, where V is the volume of the autoclave and Q is the flow rate of the subcritical water. The flow rate Q can, for example, be between 1 and 4 ml/min, preferably between 2 and 3 ml/min.

The temperature and the pressure of the water are chosen so as to keep the water in the subcritical state, more particularly to keep the water in the liquid state at a temperature of greater than 100° C. It is thus possible to use an extraction temperature of between 120 and 175° C., preferably between 150 and 175° C., for example of 150° C., and a pressure of 10 to 60 bar, preferably of 20 to 60 bar, for example of approximately 30 bar. In any case, the water is kept below the critical point, corresponding to the temperature/pressure pairing of 374° C./220 bar. This is because, beyond that, the water reaches the supercritical state. This process can, for example, be carried out for a total duration of 25 to 100 minutes.

The extract of distillation residue thus obtained is optionally dissolved in a solvent, such as glycerol, before being incorporated in the cosmetic composition according to the invention.

The combination of extracts of immortelle according to the invention is enclosed in a cosmetic composition which includes a physiologically acceptable medium, in particular a cosmetically acceptable medium, that is to say a medium which does not generate tingling or redness incompatible with a cosmetic use. This medium generally includes a fatty phase and an aqueous phase. It is preferred for the composition to be in the form of an emulsion, in particular of the oil-in-water or water-in-oil type.

When it is present, the aqueous phase includes water and optionally at least one constituent chosen from polyols and aqueous gelling agents. The water advantageously represents from 40% to 80%, for example from 50% to 70%, of the total weight of the composition. The polyol can in particular be chosen from glycerol, propylene glycol, butylene glycol, pentylene glycol and their mixtures and it can represent from 5% to 30% of the total weight of the composition.

The term “aqueous gelling agent” denotes a polymeric compound capable of immobilizing water molecules by becoming hydrated and thus increasing the viscosity of the aqueous phase. Such a gelling agent can be chosen from: polysaccharides, such as cellulose and its derivatives, modified starches, carrageenan, agar, xanthan gum and vegetable gums, such as guar gum or locust bean gum; synthetic polymers and in particular optionally crosslinked sodium acrylate homopolymers, and also acrylic copolymers, in particular copolymers of sodium acrylate and/or of alkyl (meth)acrylate and/or of hydroxyalkyl (meth)acrylate and/or of (polyethoxy)alkyl (meth)acrylate, with optionally at least one other monomer, advantageously 2-acrylamido-2-methylpropanesulfonic acid (AMPS), these copolymers optionally being crosslinked; and their mixtures.

For its part, the fatty phase can comprise one or more volatile and/or non-volatile oils. Examples of volatile oils are branched alkanes, such as isododecane, and linear C₁₀-C₁₃ alkanes. Mention may in particular be made, as non-volatile oils, of:
esters of acids and of monoalcohols chosen from: mono- and polyesters of saturated linear C₂-C₁₀ (preferably C₆-C₁₀) acids and of saturated linear C₁₀-C₁₈ (preferably C₁₀-C₁₄) monoalcohols, mono- and polyesters of saturated linear C₁₀-C₂₀ acids and of branched or unsaturated C₃-C₂₀ (preferably C₃-C₁₀) monoalcohols; mono- and polyesters of branched or unsaturated C₅-C₂₀ acids and of branched or unsaturated C₅-C₂₀ monoalcohols; mono- and polyesters of branched or unsaturated C₅-C₂₀ acids and of linear C₂-C₄ monoalcohols;
C₆-C₁₂ fatty acid triglycerides, such as caprylic and capric acid triglycerides and triheptanoin;
branched and/or unsaturated C₁₀-C₂₀ fatty acids (such as oleic and linoleic acids);
branched and/or unsaturated C₁₀-C₂₀ fatty alcohols (such as octyldodecanol and oleyl alcohol);
hydrocarbons, such as squalane (C₃₀), in particular vegetable squalane extracted from olive oil, and hemisqualane (C₁₅);
dialkyl carbonates, such as dicaprylyl carbonate and diethylhexyl carbonate;
dialkyl ethers, such as dicaprylyl ether; and
their mixtures.

Mention may also be made of vegetable oils which contain one or more of the abovementioned constituents.

Mention may in particular be made, as esters of acids and of monoalcohols, of monoesters, such as the coco-caprate and-caprylate mixture, ethyl macadamiate, the ethyl ester of shea butter, isostearyl isostearate, isononyl isononanoate, ethylhexyl isononanoate, hexyl neopentanoate, ethylhexyl neopentanoate, isostearyl neopentanoate, isodecyl neopentanoate, isopropyl myristate, octyldodecyl myristate, isopropyl palmitate, ethylhexyl palmitate, hexyl laurate, isoamyl laurate, cetostearyl nonanoate, propylheptyl caprylate and their mixtures. Other esters which can be used are the diesters of acids and of monoalcohols, such as diisopropyl adipate, diethylhexyl adipate, diisopropyl sebacate and diisoamyl sebacate.

Examples of vegetable oils are in particular wheat germ, sunflower, argan, hibiscus, coriander, grape seed, sesame, corn, apricot, castor, shea, avocado, olive, soybean, sweet almond, palm, rapeseed, cottonseed, hazelnut, macadamia, jojoba, alfalfa, poppy, red kuri squash, pumpkin, blackcurrant, evening primrose, lavender, borage, millet, barley, quinoa, rye, safflower, candlenut, passionflower, muscat rose, echium, camelina or camellia oil.

The composition according to the invention can additionally comprise at least one active agent chosen from desquamating agents, soothing agents, antimicrobial agents, antiseborrheic agents and their mixtures. In an alternative form or in addition, the composition can comprise at least one active agent chosen from probiotics, such as a bacterium of the genus Lactobacillus or Bifidobacterium; prebiotics, such as β-glucan, inulin, α-galactooligosaccharides and fructooligosaccharides; and their mixtures.

The desquamating agent can in particular be chosen from β-hydroxy acids, such as salicylic acid or an acylated derivative of salicylic acid, in particular a 5-acylsalicylic acid, such as 5-(n-octanoyl)salicylic acid, 5-(n-dodecanoyl)salicylic acid and 5-(n-decanoyl)salicylic acid, in free or salified form, in particular in the form of salts obtained by salification with an inorganic or organic base. Salicylic acid can also be used in the form of a plant extract, in particular of wintergreen (Gaultheria procumbens), containing it. Other desquamating agents which can be used are a-hydroxy acids (AHAs), such as glycolic, citric, lactic, tartaric, malic or mandelic acid; polyhydroxy acids; gentisic acid or its esters, in particular tocopherol gentisate; oligofucoses; cinnamic acid; certain derivatives of jasmonic acid; sugar derivatives, such as 6-O-octanoyl-D-maltose and N-acetylglucosamine; and their mixtures. In particular, salicylic acid can be used.

Examples of soothing agents are in particular panthenol, allantoin, β-glycyrrhetinic acid, extracts containing it (such as an extract of Glycyrrhiza glabra), plankton and their extracts, escin and plant extracts containing it (in particular of horse chestnut), aqueous, aqueous/alcoholic or aqueous/glycolic extracts of rose, cornflower, camomile, lime, peony, hawthorn, mallow, marigold, sweet clover, sage, elder, arnica, oregano, green tea, iris, water lily, birch bark, orange blossom or aloe vera, asiatic acid and extracts (in particular of Centella asiatica) containing it, extracts of algae of Laminaria type, zinc or copper pyrrolidonecarboxylate, essential oils of coriander, lemon balm, lavender, mint or camomile, ursolic acid and extracts (in particular of rosemary) containing it, sunflower unsaponifiables, fucose-containing polysaccharides, amino acids, their salts and their acylated derivatives, galactolipids and plant extracts (in particular of oats) containing them, acexamic acid, tranexamic acid and their mixtures.

Mention may in particular be made, as examples of antimicrobial agents, of undecylenic acid and its salts, phytic acid, N-acetyl-L-cysteine acid, lipoic acid, azelaic acid and its salts, arachidonic acid, resorcinol, octopirox, octoxyglycerin, caprylyl glycol, octanoyl glycine, farnesol, phytosphingosines and their mixtures.

Finally, the antiseborrheic agents comprise in particular zinc salts, such as zinc gluconate, pidolate or lactate; selenium chloride; pyridoxine or vitamin B6; capryloyl glycine, optionally as a mixture with sarcosine and an extract of Cinammomum zeylanicum, as in the product Sepicontrol® A5 from Seppic; a plant extract, in particular of rose (Rosa canina or Rosa multiflora), of Laminaria saccharina, of Spirea ulmaria, of Hypericum perforatum, of Eugenia caryophyllata, of Humulus lupus, of Mentha piperita, of Rosmarinus officinalis, of Salvia officinalis, of Thymus vulgaris, of lentils (Lens esculenta), of Enantia chlorantha or of Serenoa repens or also of the genus Silybum; and their mixtures.

These additional active agents can be present in the composition according to the invention in a content ranging from 0.001% to 20% by weight, with respect to the total weight of the composition, preferably from 0.01% to 10%, more preferentially still from 0.5% to 5% by weight, with respect to the total weight of the composition.

The composition according to the invention can additionally contain various constituents which can be dispersed in the fatty phase and/or in the aqueous phase, provided that they are compatible with topical application to the skin.

It can thus include at least one oil-in-water or water-in-oil emulsifier which is generally nonionic, such as polyoxyethylene esters, optionally polyethoxylated sorbitan esters, optionally polyethoxylated fatty acid and glycerol esters, fatty alcohol and sugar ethers, such as alkyl glucosides, sugar esters and in particular sucrose esters, and their mixtures. The emulsifiers can represent from 2% to 10% of the total weight of the composition.

The composition according to the invention can also comprise one or more pulverulent fillers, which are advantageously provided in the form of porous or hollow microparticles. These fillers are preferably porous in order to be able to absorb sebum and thus mattify the skin. They are in principle substantially spherical. These fillers can in particular be chosen from:
organic fillers, such as: powders of polysaccharides and in particular of native starch, of modified starch or of cellulose; powders of acrylic polymers, such as poly(methyl methacrylate), of polyamides or of polyolefins; powders of dried algae, such as Corallina officinalis;
inorganic fillers, such as silica, clays, perlite and talc;
and their mixtures.

The use of silica is preferred as inorganic filler.

These fillers can represent from 1% to 5% by weight, with respect to the total weight of the composition.

The composition according to the invention can additionally comprise additives chosen in particular from fragrances; sequestering agents; pH adjusters; preservatives; pigments; dyes; and their mixtures.

This composition can be provided in any form suitable for topical application to the skin and in particular in the form of a milk, cream, fluid, lotion, gel, paste or film. It is generally a leave-in composition and in particular a care composition for the skin.

In an alternative form, the composition according to the invention can be a rinse-out composition used for the care of the skin, in particular of the face. In this case, it can, for example, be used as a mask or as a scrubbing paste.

The combination of extracts of immortelle according to the invention is suitable for reducing and intended to reduce the visibility of the skin pores and/or is suitable for tightening and intended to tighten the skin pores. This expression is understood to mean the reduction in the diameter of opening of the pores and/or in the pore density and/or in the surface area of the pores, with respect to the surface area of the skin. The visibility of the pores can be assessed in vivo by scoring or by means of an image analysis device which makes it possible to extract and to quantify specific measurement parameters (surface area, density in particular) from high-resolution photographs of the faces of volunteers, the scoring or the image analysis being carried out before and after application of a composition including the combination according to the invention, as illustrated in the examples below.

The cosmetic composition may be applied to the skin of people exhibiting dilated pores, associated or not with greasy skin and/or with cutaneous signs of aging. It is generally applied to at least one area of the face, in particular to the T-zone, that is to say to the forehead, the nose and the chin, and/or to the cheeks. This composition can be applied once or more times daily, for example in the morning and/or in the evening.

In any case, the combination of extracts of immortelle according to the invention is applied to the skin in an amount sufficient to reduce the visibility of the pores. For example, an amount of 0.5 to 5 mg/cm² of cosmetic composition can be applied each day to the area of skin concerned.

It advantageously includes:
from 0.01% to 5%, preferably from 0.1% to 1%, by weight of extract of distillation residue of immortelle, and
from 0.001% to 5% by weight, preferentially from 0.001% to 1% by weight and more preferentially from 0.01% to 0.1% by weight of essential oil of immortelle, with respect to the total weight of the cosmetic composition.

### EXAMPLES

A better understanding of the invention will be obtained in the light of the following examples, which are given purely by way of illustration and are not intended to limit the scope of the invention, defined by the appended claims.

### Example 1: Preparation and Characterization of the Extracts—Comparison with an Aqueous Extract

### 1.1—Preparation of the Extracts

### a) Essential Oil of Immortelle

Flowering heads of Helichrysum italicum of Corsican origin were subjected to a conventional hydrodistillation process, in order to recover, on the one hand, an essential oil and a hydrosol and, on the other hand, a residue. The essential oil was subsequently separated from the hydrosol in a conventional way.

### b) Extract of Distillation Residue of Immortelle

A distillation residue of immortelle, consisting of the hydrodistillation residue obtained on conclusion of the extraction of the essential oil described above, was used. These distillation residue were subjected to extraction with subcritical water by percolation of hot water under pressure, in a reactor containing the dried and ground distillation residue.

The extract thus obtained was diluted in glycerin in a ratio by weight of 45:55.

### c) Aqueous Extract of Immortelle

By way of comparison, an aqueous extract of immortelle was prepared by maceration in water at 50° C. of the flowering heads of immortelle of the species Helichrysum italicum, in a plant/water ratio of 10% by weight.

### 1.2—Characterization

### a) Comparison between the Extract of Distillation Residue and an Aqueous Extract of Immortelle

### Equipment

Ultra high performance liquid chromatography (Agilent Technologies, USA)
Automatic injector 1290 series
Diode array detector (DAD) 1260 series
Mass spectrometer: Esquire 6000 (Bruker Daltonics, Bremen) equipped with an electrospray ionization (ESI) source.

### Preparation of the Samples

The aqueous extract was diluted to a 10^{th} in a MeOH/H₂O (50/50) mixture.
The extract of distillation residue according to the invention was diluted to a ½ in a MeOH/H₂O (50/50) mixture.

### Analytical Method

The solutions obtained after dilution/extraction were filtered on a PTFE filter (0.45 μm) and then injected (1 μl) onto an Agilent C18 column (2.1 mm×100 mm; 1.8 μm) at a temperature maintained at 25° C. and at a flow rate of 0.4 ml/min. The following were used as solvents: A=H₂O/HCOOH (0.1%) and B=ACN/HCOOH (0.1%). The elution gradient for the polyphenols was as follows:

TABLE 1

| Time (min.) | % Solvent B |
|---|---|
| 0 | 5 |
| 2 | 18.3 |
| 6 | 29 |
| 6.5 | 31.7 |
| 9.5 | 38.3 |
| 11.5 | 71 |
| 15 | 95 |
| 16 | 100 |
| 17 | 100 |
| 17.2 | 5 |
| 20.2 | 5 |

At the outlet of the diode array detector, the eluent was injected into the mass spectrometer. The analyses were carried out in negative or positive mode.

The LC-MS spectra were acquired over the entire range of the masses (m/z) extending from 100 to 1400. All the data was subsequently collected and processed by Hystar® software, version 3.0.

### Results

TABLE 2

| | Invention | Aqueous extract |
|---|---|---|
| Compound | (μg/ml) | (μg/ml) |
| Chlorogenic acid isomer | 80.7 ± 0.7 | 28.0 ± 1.0 |
| Chlorogenic acid | 102.9 ± 0.5 | 9.6 ± 0.3 |
| Dicaffeoylglucaric acid | 73.3 ± 0.4 | 2.4 ± 0.1 |
| Caffeic acid | 18.8 ± 0.9 | 38.1 ± 0.3 |
| Quercetagetin O-hexoside | 29.1 ± 0.3 | n.d. |
| Flavonoid O-hexoside | 19.3 ± 0.3 | n.d. |
| Dicaffeoylquinic acid | 105.0 ± 1.2 | n.d. |
| Dicaffeoylquinic acid | 31.3 ± 0.7 | <LQ |
| Dicaffeoylquinic acid | 193.2 ± 2.1 | <LQ |
| Dicaffeoylquinic acid | 268.5 ± 3.2 | n.d. |
| Leontopodic acid B | 54.5 ± 1.0 | n.d. |
| Leontopodic acid B isomer | 66.7 ± 1.1 | n.d. |
| Leontopodic acid B isomer | 13.8 ± 0.1 | n.d. |
| Quercetin O-hexoside-O- | 4.85 ± 0.03 | n.d. |
| rhamnoside | | |
| Arzanol | 12.2 ± 0.2 | n.d. |
| Methylarzanol | 0.17 ± 0.02 | n.d. |
| Dimethylarzanol | | n.d. |
| Hydroxytremetone | | Presence |

n.d.: not detected;
<LQ: detected but below the quantification limit

As emerges from this table, the extract of distillation residue used according to the invention is characterized by a high content of flavonoid heterosides, in particular of flavonol O-hexosides dihydroxylated on the B ring. They are also richer in chlorogenic acid and in dicaffeoylquinic acid than the aqueous extracts. Furthermore, it was confirmed by liquid chromatography-mass spectrometry that the “flavonoid O-hexoside” did not correspond to any other of the compounds mentioned above.

### b) Comparison with an Immortelle Hydrosol

### Procedure

The phenolic compounds present in the hydrosol obtained in 1.1a) were analyzed by high performance liquid chromatography-mass spectrometry (UPLC-DAD-MS) using an Agilent Technologies chromatograph equipped with an Esquire 6000 mass spectrometer (Bruker Daltonics, Bremen) provided with an electrospray ionization (ESI) source.

The hydrosol was diluted to 50% in a methanol/water (50/50) mixture.

The solution obtained was filtered on a PTFE filter (0.45 μm) and then injected (1 μl) onto an Agilent C18 column (2.1 mm×100 mm; 1.8 μm) at a temperature maintained at 25° C. The solvents used were: A=H₂O/0.1% HCOOH and B=ACN/0.1% HCOOH; the flow rate was 0.4 ml/min.

At the outlet of the diode array detector, the eluent was injected into the mass spectrometer. The analyses were carried out in negative or positive mode.

The data was subsequently collected and processed by Hystar® software, version 3.0.

### Results

It was observed that the hydrosol did not include quercetagetin O-hexoside or quercetin O-hexoside-O-rhamnoside, unlike the extract of the distillation residue used according to the invention.

### c) Characterization of the Essential Oil

The test described in 1.2b) was repeated on the essential oil obtained in 1.1a). To do this, 2 ml of essential oil were dissolved in 1 ml of hexane, then this oily solution was extracted three times with 2 ml of a methanol/water (60/40) mixture. The aqueous/alcoholic phases were combined.

Here again, it was observed that the essential oil of immortelle did not include quercetagetin O-hexoside or quercetin O-hexoside-O-rhamnoside.

More generally, it was demonstrated that the essential oil of immortelle was devoid of polyphenols and included terpenoids, in particular neryl acetate (more than 30% by weight), eudesmol and curcumene.

### Example 2: Effect of the Combination of Extracts of Immortelle According to the Invention on the Visibility of Pores

### 2.1—Equipment and Method

A clinical study was carried out on a panel of 33 healthy adult women, aged from 37 to 63 years, exhibiting dilated pores on the cheeks, who were asked to apply the study product at home on half the face, twice daily (morning and evening). A control product was applied on the other half of the face at the same frequency. The side on which the two different formulae were applied was defined using a specific randomization.

The test product consisted of a cosmetic composition including the combination of extracts of immortelle according to the invention (0.5% of extract of distillation residue of immortelle and 0.03% of essential oil of immortelle). The control product corresponded to the same composition, but lacked these two extracts. The formula base common to these products was the following composition:

| | |
|---|---|
| Hydrophilic gelling agent | 1.00% |
| Sodium gluconate | 0.20% |
| Ethylhexylglycerin | 0.20% |
| Oil | 3.00% |
| Preservative | q.s. |
| Water | q.s. for 100% |

Photographs were taken at different time intervals at an ISO9001-certified investigation center, using the VISIA-CR image acquisition device (supplied by Canfield).

The images were taken specifically:
at d0, before the first application of the product,
at d28, after 28 days of application (±2 days),
at d56, after 56 days of use (±2 days).

To do this, the volunteers were first subjected to a period of acclimatization of 20 minutes at the investigation center, in a room having controlled temperature (22° C.±2° C.) and controlled humidity (45%±10%). Photographs were subsequently taken of their entire faces (eyes closed) in standard, cross-polarized and parallel-polarized lighting mode.

The photographs which were taken were subsequently analyzed, focusing on the pores located on each cheek close to the nasolabial fold. The volume of the pores was analyzed at each visit of the volunteer. It is defined as the product of the surface area of the pore by the depth of the pore, where the surface area of the pore corresponds to the number of pixels in the area under consideration and the depth of the pore corresponds to the difference in intensity between the area under consideration and the surrounding area.

A statistical analysis of the results was carried out using the Wilcoxon test.

### 2.2—Results

The difference in volume of the pores after 28 days was compared between the half-face treated with the composition according to the invention and the half-face treated with the control composition.

The difference in volume measured was significant (p=0.041) and observable with the naked eye by the sponsor of the study.

In addition, the composition according to the invention was well tolerated, no undesirable effect having been reported by the volunteers.

### Example 3: Cosmetic Compositions

The following compositions are prepared in a way conventional to a person skilled in the art, by mixing the ingredients below (identified in capital letters by their INCI names) in the proportions by weight indicated.

Face cream

| | |
|---|---|
| O/W emulsifiers | 6.00% |
| Fatty substances | 15.00% |
| Hydrophilic gelling agents | 3.00% |
| Polyols | 3.50% |
| Antiaging active agents | 5.50% |
| Antiseborrheic active agents | 3.00% |
| Sodium hydroxide | 1.00% |
| Essential oil of immortelle | 0.03% |
| Extract of distillation residue of immortelle | 0.50% |
| Fragrances | q.s. |
| Preservatives | q.s. |
| Antioxidants | q.s. |
| Water q.s. for | 100.00% |

Serum

| | |
|---|---|
| O/W emulsifiers | 1.00% |
| Fatty substances | 8.00% |
| Hydrophilic gelling agents | 3.00% |
| Polyols | 6.00% |
| Antiaging active agents | 5.00% |
| Antiseborrheic active agents | 4.00% |
| Essential oil of immortelle | 0.04% |
| Extract of distillation residue of immortelle | 1.00% |
| Fragrances | q.s. |
| Preservatives | q.s. |
| Antioxidants | q.s. |
| Water q.s. for | 100.00% |

Seboregulatory mask

| | |
|---|---|
| GLYCERIN | 10.0% |
| ZINC GLUCONATE | 0.2% |
| CETEARYL ALCOHOL & CETEARYL GLUCOSIDE | 3.0% |
| MYRISTYL ALCOHOL & MYRISTYL GLUCOSIDE | 2.0% |
| OCTYLDODECANOL | 3.0% |
| PENTYLENE GLYCOL | 1.0% |
| SODIUM POLYACRYLATE | 0.5% |
| SODIUM STEAROYL GLUTAMATE | 0.3% |
| ACRYLATE/C10-30 ALKYL ACRYLATE | |
| CROSSPOLYMER | 0.2% |
| XANTHAN GUM | 0.1% |
| DISODIUM EDTA | 0.1% |
| Essential oil of immortelle | 0.02% |
| Extract of distillation residue of immortelle | 0.4% |
| Fragrance | q.s. |
| AQUA q.s. for | 100% |

1-11. (canceled)

12. A cosmetic method for reducing the visibility of skin pores and/or tightening skin pores, comprising the topical application to the skin of a cosmetic composition including the combination of an essential oil of immortelle of the species Helichrysum italicum and of an extract of distillation residue of immortelle of the species Helichrysum italicum obtained by extraction of the distillation residue with subcritical water.

13. The cosmetic method as claimed in claim 12, wherein the cosmetic composition includes: (a) from 0.01% to 5% by weight of extract of distillation residue of immortelle and (b) from 0.001% to 5% by weight of essential oil of immortelle, with respect to the total weight of the cosmetic composition.

14. The cosmetic method as claimed in claim 12, wherein the cosmetic composition is applied to the skin of the face.

15. The cosmetic method as claimed in claim 12, wherein the extract of distillation residue of immortelle includes flavonol heterosides, the B ring of which is dihydroxylated.

16. The cosmetic method as claimed in claim 12, wherein the extract of distillation residue of immortelle additionally includes chlorogenic acid and leontopodic acid B, in a ratio by weight of flavonol heterosides, the B ring of which is dihydroxylated, to chlorogenic acid of between 0.05:1 and 1:1 and in a ratio by weight of flavonol heterosides, the B ring of which is dihydroxylated, to leontopodic acid B of between 0.05:1 and 1:1.

17. The cosmetic method as claimed in claim 12, wherein the flavonol heterosides comprise at least one quercetagenin heteroside, at least one quercetin heteroside or mixtures thereof.

18. The cosmetic method as claimed in claim 12, wherein the extract of distillation residue of immortelle includes at least 30 μg/ml of flavonol heterosides, the B ring of which is dihydroxylated.

19. The cosmetic method as claimed in claim 12, wherein the volatile compounds present in the extract of distillation residue of immortelle include more than 30% of ketones and less than 2% of nerol.

20. The cosmetic method as claimed in claim 12, wherein the essential oil of immortelle and/or the extract of distillation residue of immortelle result from immortelle of the species Helichrysum italicum of Corsican origin.

21. The cosmetic method as claimed in claim 12, wherein the essential oil of immortelle includes, as main constituent, neryl acetate, which represents at least 30% by weight of the total constituents of the essential oil, as measured by gas chromatography coupled with an analysis by mass spectrometry (GC-MS).

22. The cosmetic method as claimed in claim 13, wherein the cosmetic composition includes: (a) from 0.1% to 1%, by weight of extract of distillation residue of immortelle and (b) from 0.01% to 0.1% by weight of essential oil of immortelle, with respect to the total weight of the cosmetic composition.

23. The cosmetic method as claimed in claim 16, wherein the extract of distillation residue of immortelle additionally includes chlorogenic acid and leontopodic acid B, in a ratio by weight of flavonol heterosides, the B ring of which is dihydroxylated, to chlorogenic acid of between 0.1:1 to 0.5:1, and in a ratio by weight of flavonol heterosides, the B ring of which is dihydroxylated, to leontopodic acid B of between 0.1:1 and 0.5:1.

24. The cosmetic method as claimed in claim 17, wherein the flavonol heterosides comprise at least one quercetagenin O-hexoside, at least one quercetin O-hexoside-O-rhamnoside, or mixtures thereof.

25. The cosmetic method as claimed in claim 14, wherein the cosmetic composition is applied to the forehead, the nose, the chin and/or the cheeks.
